(19) United States
(12) Patent Application Publication
(10) Pub. No.: US 2008/0125414 A1
(43) Pub. Date: May 29, 2008

(54) Method of Treatment

(75) Inventor: Karen Jackson, Deepcar Sheffield (GB)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(73) Assignee: Panos Therapeutics Limited, Oxford (GB)

(21) Appl. No.: 11/852,727

(22) Filed: Sep. 10, 2007

Related U.S. Application Data
(63) Continuation-in-part of application No. 10/622,492, filed on Jul. 21, 2003, now abandoned.
Application No. 10/622,492 is a continuation-in-part of application No. 10/349,431, filed on Jan. 22, 2003, now Pat. No. 6,713,470.
Application No. 10/349,431 is a continuation-in-part of application No. 10/108,659, filed on Mar. 27, 2002, now abandoned.
Application No. 10/108,659 is a continuation-in-part of application No. 10/053,962, filed on Jan. 22, 2002, now abandoned.

(51) Int. Cl. A61K 31/5513; A61P 43/00

(52) U.S. Cl. 514/221

(57) ABSTRACT

A method of treating a patient undergoing analgesic therapy which comprises the separate, simultaneous or sequential administration of a therapeutically effective amount of an analgesic and an analgesic sparing amount of devazepide.

There is also described the use of devazepide in the manufacture of a medicament which reduces the dose required for administration of an opioid analgesic and superpotentiates the effect of the analgesic.

The present application is a Continuation-in-part of application Ser. No. 10/622,492, filed on Jul. 21, 2003, which is a Continuation-in-part of application Ser. No. 10/349,431, filed on Jan. 22, 2003, now U.S. Pat. No. 6,713,470, which is a Continuation-in-part of U.S. application Ser. No. 10/108,659, filed on Mar. 27, 2002, now abandoned, which is a Continuation-in-part of Ser. No. 10/053,962, filed on Jan. 22, 2002, now abandoned, the entire disclosures of which are incorporated by reference herein.

This invention relates to a novel method of treatment and to a novel use of a medicament.

Faris, in Science 1984; 226:1215-1217 describes a reduction of morphine analgesia by endogenously released cholecystokinin. Thus, Faris suggests that concomitant administration of morphine and a specific CCK antagonist may allow reduction in the initial dose of morphine required and a reduction in the frequency of its administration.

International Patent Application No. WO 99/18967 describes pharmaceutical compositions for treating chronic and neuropathic pain which comprises an analgesic amount of an opioid and an opioid potentiating amount of a CCK antagonist. WO '967 describes the use of both CCK-A (CCK-1) antagonists and CCK-B (CCK-2) antagonists, although it is described that, generally, CCK-B (CCK-2) antagonists are preferred. Moreover, page 2, lines 6 to 8 of WO '967 describes that CCK-A (CCK-1) antagonists may be suitable, but only at relatively higher dosages.

One specific CCK-A (CCK-1) antagonist which is mentioned is devazepide, which is 3s-(−) 1,3dihydro-3-(2-indolecarbonylamino)-1-methyl-5-phenyl-2H-1,4-benzodiazepin-2-one.

International Patent Application No. WO 99/18967 describes a pharmaceutical formulation comprising a CCK antagonist, such as devazepide (Devacade®), an opioid and a biphasic carrier, comprising a glyceride derivative organic phase. The use of the CCK antagonist is intended to block the CCK receptors thereby reversing or preventing the development of opioid tolerance in patients and potentiating the analgesic effect of the opioid.

Generally, patients undergoing opioid therapy will receive a stable dose of an opioid. A patient may also use an additional ‘breakthrough pain’ relieving dose of opioid therapy, as symptoms dictate.

As hereinbefore described, International Patent Application No. WO 99/18967 discloses the administration of devazepide in combination with an opioid analgesic so as to potentiate the analgesic effect of the overall dose of the opioid. In practice the expected outcome would result in the analgesia, or the “pain level”, experienced by a patient generally remaining the same, whilst the dosage of opioid administered to the patient is reduced or analgesia improving with the same dose of opioid. However, we have now found that in fact devazepide has a “superpotentiating” effect in combination with an opioid. Thus, in the present invention the use of devazepide can act to enable the overall dose of opioid to be reduced or minimised, concurrent with improved analgesia, i.e. an increase in the analgesia, or a lowering of “pain level”, experienced by the patient. Indeed clinical studies have shown that the opioid dose may be reduced as much as 95% in some cases, in others as much as 75%. Thus, the administration of a combination of an opioid and devazepide does not just retain the “pain level” experienced by a patient, but actually improves it, i.e. it is not just a case of opioid sparing or opioid potentiation, but the effect of the opioid is superpotentiated.

Thus according to the invention we provide a method of treatment of a patient undergoing analgesic therapy by the administration of a therapeutically effective amount of an analgesically active medicament which method comprises the separate, simultaneous or sequential administration of an analgesic sparing amount of devazepide.

The analgesic sparing amount of devazepide will advantageously cause a reduction in the amount of analgesic administered to the patient. The reduction may vary but may, for example, be a reduction of from 25% to 95% w/w of the amount of analgesic required in the absence of devazepide, more preferably from 25% to 75% w/w.

Thus according to a further aspect of the invention we provide a method of treatment of a patient as hereinbefore described characterised in that a reduced amount of analgesic of from 25% to 95% w/w may be administered. In a preferred embodiment of the invention a reduced amount of analgesic of from 25 to 75% w/w may be administered.

In the method of the invention a variety of opioids may be used. Thus, the opioid may be selected from those which are effective analgesics and particularly those which need to be administered at relatively high or increasing doses. Examples include morphine, or a salt thereof such as the sulphate, chloride or hydrochloride other analgesics such as, meperidine, pentazocine, dextropropoxyphene, pethidine, fentanyl, alfentanil, alphaprodine, dextromoramide, diphenoxylate, dipipanone, meptazinol, methadone, nalbuphine, phenadoxone, phenazocine, remifentanil, tramadol, or the 1,4-hydroxymorphinan opioid analgesics such as butorphanol, morphine-6-glucuronide, codeine, dihydrocodeine, diamorphine, buprenorphine, heroin (diacetylmorphine), hydrocodone (dihydrocodeinone), hydromorphone (dihydromorphinone), levorphanol, metopon (methyldihydromorphinone), oxycodone (dihydrohydroxycodeinone), oxymorphone (dihydrohydroxymorphinone); or a salt of any of the aforementioned. Naloxone is also included within the definition of an opioid. Especially preferred analgesics which may be mentioned are hydromorphone, oxycodone, morphine, e.g. morphine sulphate and fentanyl. In a preferred embodiment of the invention the analgesic is morphine or morphine sulphate. In a further preferred embodiment the opioid is fentanyl, or a salt thereof.

According to a further feature of the invention we provide a method of treatment of a patient requiring analgesia which comprises the administration of a therapeutically effective amount of an analgesic whilst minimising the amount of the analgesic by the separate, simultaneous or sequential administration of a therapeutically effective amount of devazepide.

In a preferred aspect of the invention the analgesic will be an opioid analgesic.

According to a further aspect of the invention we provide the use of devazepide in the manufacture of a medicament which reduces the dose of the analgesic and superpotentiates the analgesic effect.

We further provide the use of devazepide in the manufacture of a medicament for the treatment of analgesia wherein the treatment comprises the administration of a therapeutically effective amount of an analgesic whilst minimising the amount of the analgesic by the separate, simultaneous or sequential administration of a therapeutically effective amount of devazepide.

In the method of the invention the devazepide and/or the opioid may be administered using any methods conventionally known per se. Thus, such methods would include, but shall not be limited to, administration intravenously, intra-arterially, orally, intrathecally, intranasally, intrarectally, intramuscularly/subcutaneously, by inhalation and by transdermal patch. When the devazepide and/or opioid is administered intravenously, it may, for example, be as an intravenous bolus or a continuous intravenous infusion. When the devazepide and/or the opioid is administered subcutaneously, it may, for example, be by subcutaneous infusion. Preferably, the opioid and/or devazepide are administered intravenously or orally. Oral administration is especially preferred. Preferentially, the opioid and the devazepide will be administered using the same mode of administration. Thus, for example, when the opioid is administered intravenously then the devazepide will be administered intravenously also. Similarly, when the opioid is administered orally then the devazepide will be administered orally also. However, it is within the scope of the invention for either the opioid to be administered orally and the devazepide to be administered intravenously or vice versa. In a further preferred embodiment the opioid may be administered by a transdermal patch. When a transdermal patch is used, the preferred opioid is fentanyl.

According to a further aspect of the invention we provide the use of devazepide in the manufacture of a pharmaceutical composition as hereinbefore described.

Thus, for example, in one embodiment of the invention the composition may be made up into a capsule formulation, e.g. with a fill weight of 150 mg±5% by weight or 300 mg±5% by weight. In the one preferred embodiment, the capsule formulation may comprise 1.25 mg devazepide, and 148.75 mg of a filler or other excipient, e.g. corn starch. In a further preferred embodiment, the capsule formulation may comprise 2.5 mg devazepide, and 297.5 mg of a filler or other excipient, e.g. corn starch.

Thus, such fillers may be selected from the group lactose, mannitol, talc, magnesium stearate, sodium chloride, potassium chloride, citric acid, spray-dried lactose, hydrolysed starches, directly compressible starch, microcrystalline cellulose, cellulosics, sorbitol, sucrose, sucrose-based materials, icodextrin, calcium sulphate, dibasic calcium phosphate and dextrose. A preferred filler is starch, e.g. corn starch.

When the composition of the invention includes a filler, the size of the devazepide and filler particles may be the same or different. However, in a preferred embodiment the sizes of the devazepide and filler particles will differ. Preferentially, the devazepide and/or the filler may be of reduced particle size, e.g. by milling.

Thus, in the method of the invention the daily dosage of devazepide may vary depending upon, inter alia, the weight of the patient, the method of administration, etc. In patients that are suffering serious disorders, such as cancer patients, the weight of the patient may be very low and therefore the dosage of devazepide consequentially may be low. Thus the daily dosage of devazepide may be up to 0.7 mg/kg/day. Preferably, the daily dosage of devazepide may be from 25 μg/kg/day to 0.7 mg/kg/day, more preferably from 50 μg/kg/day to 0.5 mg/kg/day. For oral administration the daily dosage of devazepide may be from preferably 0.07 mg/kg/day to 0.7 mg/kg/day. More preferably, for oral administration the daily dosage of devazepide may be from 0.07 mg/kg/day to 0.29 mg/kg/day. For intravenous administration the dosage of devazepide is preferably 50 μg/kg/day to 0.5 mg/kg/day.

In particular, the ratio of devazepide to dose of opioid may be varied. Thus, the ratio devazepide to opioid may be from 2:1 to 1:200 w/w, preferably from 1:2 to 1:40 w/w.

In the method of the invention the dosage of the opioid analgesic administered may vary depending upon, inter alia, the nature of the opioid analgesic, the weight of the patient, the method of administration, etc. Thus, for example, the dosage of, e.g. an opioid, such as morphine, may be from 5 to 2000 mg daily. A particular dosage which may be mentioned is from 10 to 240 mg daily. A daily dosage of morphine may be from 5 to 100 mg or occasionally up to 500 mg.

A further aspect of the invention is that upon subsequent treatments over a period of at least 8 weeks, e.g. at least 7 weeks, at least 6 weeks, or at least 5 weeks, the amount of opioid is reduced by an amount from about 25% to about 95% by weight of the amount of the opioid required in the absence of devazepide and wherein the analgesia experienced by the patient is increased. In one preferred embodiment, the invention relates to a method of reducing the amount of an opioid administered to a patient requiring neuropathic pain relief, comprising: administering to the patient a therapeutically effective amount of the opioid and a potentiating amount of devazepide, wherein upon subsequent treatments over a period of at least 8 weeks, the amount of the opioid is reduced by an amount of from about 25% to about 95% by weight of the amount of the opioid required in the absence of the devazepide and wherein the analgesia experienced by the patient is increased. The opioid is optionally administered at regular intervals to achieve stabilized medication and wherein the devazepide is administered on a daily basis. In some embodiments, the devazepide is administered twice daily.

The devazepide used in the method of the invention is the S enantiomer, preferentially, the S enantiomer wherein the level of R enantiomer, which may be present as an impurity, is not greater than 1.5% w/w.

The invention will now be illustrated by way of example only.

### EXAMPLE 1

### Clinical Crossover Study

A multi-centre, double blind placebo controlled crossover study was designed to investigate devazepide as adjunctive therapy to strong opioids in patients with moderate or severe neuropathic pain.

### 1.1 Rationale for Study Design

The study was conducted to observe the effects of two different doses for devazepide (1.25 mg b.d. and 5 mg b.d.) in patients when given as an adjunct to strong opioids. Previous studies have shown devazepide is well tolerated at levels up to 5 mg bd. This study was designed to further investigate the analgesic effect and the safety and toxicity of devazepide compared to placebo when administered twice daily for two weeks.

### 1.2 Pre-Treatment

Patients completed a pre-treatment period of at least two weeks duration. During pre-treatment the patients continued to take regular doses of strong opioids and breakthrough analgesics when required. All use of strong opioids and analgesics was recorded. Patients completed an assessment of their pain each evening prior to going to bed, using a pain questionnaire. Any adverse events and changes in concomitant medication were also recorded.

### 1.3 Treatment

Following the two-week pre-treatment period, each patient received blinded treatments of devazepide 1.25 mg, devazepide 5 mg and placebo twice daily for two weeks with washout periods between each treatment, according to the following schedule.

TABLE I
Treatment Schedule

| Study | Pre- | | | | | |
|---|---|---|---|---|---|---|
| period | treatment | Treatment 1 | Washout | Treatment 2 | Washout | Treatment 3 |
| Number of | 2 weeks | 2 weeks | 4-21 days | 2 weeks | 4-21 days | 2 weeks |
| weeks or | | | | | | |
| days | | | | | | |

On each day of treatment, assessment of pain, general activity and sleep interference were made by the patients and recorded on diary cards. At the end of each treatment period, the patient rated overall pain relief using a descriptive scale, and the opinion of both the patient and investigator of that treatment was recorded. All concomitant medication use (including strong opioid and breakthrough analgesic use) and adverse events were recorded throughout the study. Blood samples were collected at screening and at the end of each treatment for safety monitoring.

### 1.4 Results

Of the 62 patients screened, 41 completed the study according to the protocol. There were 18 (44%) women and 23 (56%) men, who had a mean (range) age of 48.6 (23.2 to 70.7) years. Patients were stabilised on morphine (34), oxycodone (2), fentanyl (3), methadone (1), hydromorphone (1) and diamorphine (1); with one patient taking both morphine and fentanyl. Breakthrough analgesic product use included (in addition to the above) tramadol, diclofenac, dihydrocodeine, paracetamol, codeine, aspirin, ibuprofen, dextropropoxyphene, buprenorphine, pethidine, carbamazepine, and hydromorphone (and combination products containing the above, for example; Anadin™ (aspirin), Anadin™ extra (aspirin and paracetamol), co-codamol, co-dydramol, co-proxamol, and remedeine).

The analysis of specific use of breakthrough analgesia as opposed to regular opioid use was not possible because patients were taking a variety of different analgesic drugs (including opioids) in addition to their regular opioid. It was also difficult to define breakthrough analgesia in those patients who were using the same opioid for both regular use and for breakthrough analgesia (latter to control breakthrough pain). For this reason, all analgesic medication was included in the determination of a total dose for the treatment period, irrespective of whether it was used as regular stable medication or for breakthrough pain.

The results are illustrated in Table II.

TABLE II
Analgesic use expressed as a percentage of pre-treatment use

| | | | SUM OF DAILY DOSE | | | |
|---|---|---|---|---|---|---|
| PATIENT | | | Devazepide | Devazepide | | pre |
| NUMBER | DRUG | UNITS | 1.25 mg | 5 mg | placebo | treatment |
| ap06/05 | morphine | mg | 104 | 100 | 104 | 100% |
| | tramadol | mg | 79 | 54 | 79 | 100% |
| gg01/01 | co-codamol | tablets | 106 | 78 | 67 | 100% |
| | fentanyl | mcg/h | 100 | 67 | 83 | 100% |
| gg03/03 | morphine | mg | 55 | 93 | 97 | 100% |
| ng02/02 | diclofenac | mg | 17 | 50 | 50 | 100% |
| | morphine | mg | 87 | 84 | 87 | 100% |
| ng05/05 | morphine | mg | 104 | 100 | 104 | 100% |
| | paracetamol | mg | 48 | 14 | 43 | 100% |
| sj03/03 | morphine | mg | 93 | 97 | 93 | 100% |
| | morphine | ml | 13 | 5 | 42 | 100% |
| sj10/73 | dihydrocodeine | mg | 100 | 50 | 100 | 100% |
| | morphine | mg | 100 | 98 | 100 | 100% |
| | paracetamol | mg | 75 | 50 | 100 | 100% |

### EXAMPLE 2

### 2.1 Clinical Assessment Study

A research programme has included a double blind, double dummy, randomised, crossover study of a single dose of either 1.25 mg devazepide, 5.0 mg devazepide or placebo. Patients who took part in the study had pain with a neuropathic element, and were taking regular doses of strong opioids. Following completion of the study those patients who, in the opinion of their Clinical Investigator, had gained benefit from participation were given the opportunity to consent to continue receiving devazepide treatment for a period of up to six months.

### 2.2 Study Design

This continuation study was a multicentre, open label study of devazepide at twice daily doses of 1.25 mg, 2.5 mg and 5.0 mg.

### 2.3 Study Objective

The primary objective of this study was to compare descriptive and visual analogue scale (VAS) assessments of pain and pain relief in patients with neuropathic pain.

### 2.4 Methods

At the end of the previous randomised trial, patients received 1.25 mg devazepide twice daily for an initial period of one week. After this initial one week period, the dose of devazepide was reviewed and increased, if necessary, to 2.5 mg twice daily and thereafter to 5.0 mg twice daily as required. Devazepide treatment was continued for a period of up to six months.

During the study patients were required to remain on regular doses of opioids at a dose prescribed by the investigator.

### 2.5 Study Assessments

Patients were assessed at clinic visits at week 1, week 2 (dose escalation) and thereafter at routine monthly clinic visits.

At weekly intervals for the first eight weeks and at monthly intervals thereafter, patients recorded pain and global pain relief using VAS and descriptive pain questionnaires. The questionnaires were returned to the Investigator at the monthly visits.

At each clinic visit the Investigator assessed safety and the patients' pain relief, reviewed the dosage, and decided if devazepide treatment should be continued.

### 2.6 Results

Seventeen patients elected to stay on devazepide by entering the continuation study and received devazepide at 1.25 mg, 2.5 mg or 5.0 mg twice daily for up to 26 weeks.

Of these patients, ten appeared to achieve long-term pain relief (5-26 weeks) with devazepide. Despite the requirement to remain on stable, regular doses of opioids at the dose prescribed by the investigator, several patients reduced markedly reduced their daily opioid dose.

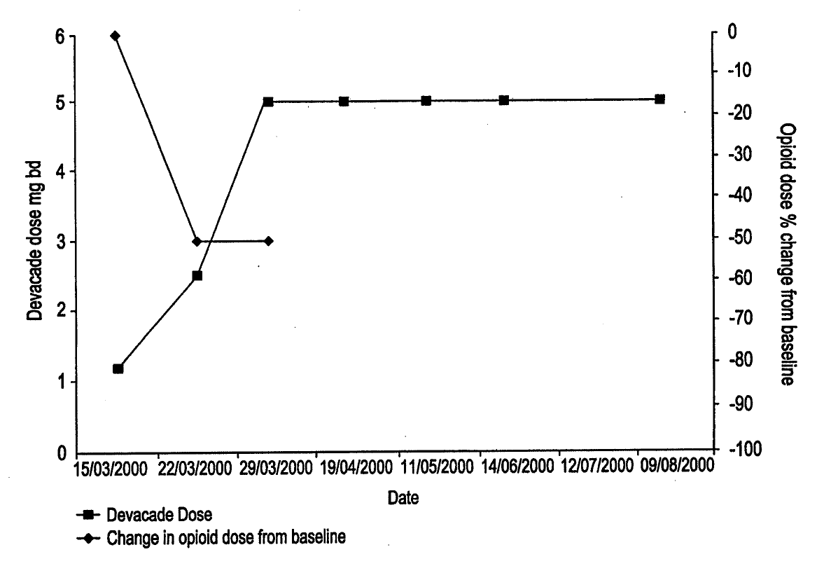

FIG. 1 illustrates the trend with a weaker opioid, dihydrocodeine. The patient(s) commenced on 120 mg dihydrocodeine per day.

1. A method of reducing the amount of an opioid administered to a patient requiring neuropathic pain relief, comprising:
administering to the patient a therapeutically effective amount of the opioid and a potentiating amount of devazepide,
wherein upon subsequent treatments over a period of at least 8 weeks, the amount of the opioid is reduced by an amount of from about 25% to about 95% by weight of the amount of the opioid required in the absence of the devazepide and wherein the analgesia experienced by the patient is increased.

2. The method according to claim 1, wherein the opioid and the devazepide are administered separately.

3. The method according to claim 1, wherein the opioid is administered at regular intervals to achieve stabilized medication and wherein the devazepide is administered on a daily basis.

4. The method according to claim 3 wherein the devazepide is administered twice daily.

5. The method according to claim 1, wherein the opioid is selected from the group consisting of morphine, meperidine, pentazocine, dextropropoxyphene, pethidine, fentanyl, alfentanil, alphaprodine, dextromoramide, diphenoxylate, dipipanone, meptazinol, methadone, nalbuphine, phenadoxone, phenazocine, remifentanil, tramadol;
butorphanol, morphine-6-glucuronide, codeine, dihydrocodeine, diamorphine, buprenorphine, heroin (diacetylmorphine), hydrocodone (dihydrocodeinone), hydromorphone (dihydromorphinone), levorphanol, metopon (methyldihydromorphinone), oxycodone (dihydrohydroxycodeinone), oxymorphone (dihydrohydroxymorphinone), and a salt of any of the aforementioned.

6. The method according to claim 1, wherein the opioid is selected from the group consisting of hydromorphone, oxycodone, morphine, fentanyl and salts thereof.

7. The method according to claim 6, wherein the opioid comprises fentanyl or a salt thereof.

8. The method according to claim 6, wherein the opioid comprises morphine or morphine sulphate.

9. The method according to claim 1, wherein the ratio of devazepide to opioid is from 2:1 to 1:400 w/w.

10. The method according to claim 9, wherein the ratio of devazepide to opioid is from 2:1 to 1:200 w/w.

11. The method according to claim 10, wherein the ratio of devazepide to opioid is from 1:2 to 1:40 w/w.

12. The method according to claim 1, wherein the opioid is fentanyl, or a salt thereof.

13. The method according to claim 1, wherein the daily dosage of devazepide is up to 0.7 mg/kg/day.

14. The method according to claim 13, wherein the daily dosage of devazepide is from 25 μg/kg/day to 0.7 mg/kg/day.

15. The method according to claim 14, wherein the daily dosage of devazepide is from 50 μg/kg/day to 0.5 mg/kg/day.

16. The method according to claim 14, wherein the devazepide is administered orally and the daily dosage of devazepide is from 0.07 mg/kg/day to 0.29 mg/kg/day.

17. The method according to claim 13, wherein the devazepide is administered intravenously at a dosage of from 50 μg/kg/day to 0.5 mg/kg/day.

18. The method according to claim 1, wherein the dosage of the opioid is from 5 to 2000 mg daily.

19. The method according to claim 18, wherein the dosage of the opioid is from 10 to 240 mg daily.

20. The method according to claim 19, wherein the dosage of the opioid is from 5 to 100 mg daily.

21. The method according to claim 1, wherein the devazepide is provided as a composition incorporating a filler or other excipient.

22. The method according to claim 21, wherein the composition is filled into a capsule.

23. The method according to claim 22, wherein the capsule formulation comprises 1.25 mg devazepide or 2.5 mg devazepide.

24. The method according to claim 22, wherein the 1.25 mg or 2.5 mg of devazepide is delivered at least twice daily.

25. The method according to claim 22, wherein the capsule containing 1.25 mg. devazepide has a fill weight of 150 mg±5% by weight and the capsule containing 2.5 mg devazepide has a fill weight of 300 mg±5% by weight.
